(19) United States
(12) Patent Application Publication
(10) Pub. No.: US 2026/0137325 A1
(43) Pub. Date: May 21, 2026

(54) BIOSIGNAL MEASUREMENT SYSTEM

(71) Applicant: NTT, Inc., Tokyo (JP)

(72) Inventors: Kento Watanabe, Tokyo (JP); Kenichi Matsunaga, Tokyo (JP)

(21) Appl. No.: 19/121,344

(22) PCT Filed: Oct. 25, 2022
(86) PCT No.: PCT/JP2022/039662

§ 371 Date: Apr. 15, 2025

(51) Int. Cl. A61B 5/308; A61B 5/00; A61B 5/273; A61B 5/282; A61B 5/30

(52) U.S. Cl. CPC A61B 5/308; A61B 5/0006; A61B 5/273; A61B 5/282; A61B 5/303; A61B 5/7203

(57) ABSTRACT

An embodiment includes a sensor device including an amplifier configured to amplify a biopotential detected by an electrode, a transmitter configured to modulate a carrier wave according to the biopotential and transmit the modulated signal wirelessly to the other sensor device, a receiver configured to demodulate the modulated signal transmitted from the other sensor device and extract information on the biopotential, a reference potential generator configured to generate a reference potential of the amplifier, and a correction circuit configured to correct an oscillation frequency of a voltage-controlled oscillator used for demodulation by the receiver.

### CROSS-REFERENCE TO RELATED APPLICATIONS

This application is a national phase entry of PCT Application No. PCT/JP2022/039662, filed on Oct. 25, 2022, which application is hereby incorporated herein by reference.

### TECHNICAL FIELD

The present invention relates to a biosignal measurement system that measures biosignal such as electrocardiogram signals.

### BACKGROUND

In the measurement of an electrocardiogram which is a kind of biopotential measurement, a potential difference between electrodes arranged on both the right and left sides of a human body is measured. The biosignal measurement system is installed in compression wear or the like worn by a person. In the example shown in FIG. 22, a device 301 is attached to a portion of compression wear 302 corresponding to the central portion of the body, and electrodes 304 provided to be in contact with the left and right waist portions are connected to the device 301 by wirings 303 laid on the compression wear 302 (NPL 1).

When the electrodes are attached to the body of a measurement subject as in the example shown in FIG. 22, the measurement subject feels discomfort due to the feeling of pressure, mounting the electrodes takes much time and effort, which is troublesome for the measurement subject. For this reason, for example, an electrode may be attached to the limbs as a place other than the trunk. However, when the electrodes are attached to the right hand and left hand of the measurement subject or when the electrodes are attached to the right foot and left foot of the measurement subject, the wiring connecting the right and left electrodes is required, and therefore there is a possibility of the activity of the measurement subject being limited.

If the wiring can be eliminated, the discomfort of the measurement subject due to the wiring can be reduced, and the restraint of the body of the measurement subject can be eliminated. When the devices are separated by eliminating wiring, it is important that the references for measuring the potentials of the right and left devices be made common. When the reference potentials do not coincide with each other, measurement accuracy may deteriorate and biopotential measurement may become difficult.

### CITATION LIST

### Non Patent Literature

[NPL 1] Nahoko Kasai et al., “Development of Functional Textile “hitoe”: Wearable Electrodes for Monitoring Human Vital Signals,” Institute of Electronics, Information and Communication Engineers, Communication Society Magazine, Vol. 11, No. 1, pp. 17-23, 2017.

### SUMMARY

Embodiments of the present invention has been made to solve the foregoing problems, and an object of embodiments of the present invention is to provide a biosignal measurement system that can easily measure biopotential in a form that eliminates wiring and is separated into two devices.

### Solution to Problem

A biosignal measurement system of embodiments of the present invention includes a first sensor device configured to be attached to one of a right-side portion and a left-side portion of a measurement subject; and a second sensor device configured to be attached to the other of the right-side portion and the left-side portion, wherein each of the first sensor device and the second sensor device includes: a first electrode configured to be in contact with the skin of the measurement subject; an amplifier configured to amplify a biopotential detected by the first electrode; a transmitter configured to modulate a carrier wave according to the biopotential amplified by the amplifier and wirelessly transmit the modulated signal to the other sensor device; a receiver configured to demodulate the modulated signal transmitted from the other sensor device and extract information on the biopotential; a reference potential generator configured to generate a reference potential for the amplifier based on the biopotential amplified by the amplifier and the biopotential output from the receiver; and a correction unit configured to correct at least one of an oscillation frequency of a voltage-controlled oscillator used for modulation by the transmitter and an oscillation frequency of a voltage-controlled oscillator used for demodulation by the receiver.

### Advantageous Effects

According to embodiments of the present invention, by connecting the first sensor device and the second sensor device through wireless communication, wiring connecting the first sensor device and the second sensor device can be eliminated. As a result, it is possible to reduce the discomfort of the measurement subject due to the wiring, and it is also possible to eliminate the physical restraint of the measurement subject. Further, in embodiments of the present invention, by providing the correction unit, at least one of the oscillation frequencies of the voltage-controlled oscillator used for modulation by the transmitter and the oscillation frequency of the voltage-controlled oscillator used for demodulation by the receiver can be corrected. In this way, it is possible to minimize variations in the oscillation frequencies of the transmitter and receiver due to influences from the external environment, and to make the reference potentials for measuring the potentials of the first and second sensor devices common. Therefore, the measurement accuracy of biopotential can be improved.

### BRIEF DESCRIPTION OF THE DRAWINGS

FIG. 1 is a block diagram showing the configuration of a biosignal measurement system according to a first embodiment of the present invention.

FIG. 2 is a circuit diagram showing the configuration of the amplifier according to the first embodiment of the present invention.

FIG. 3 is a diagram showing an example of individual differences in the oscillation frequency of a VCO.

FIG. 4 is a diagram showing the basic configuration of an FM receiver according to the first embodiment of the present invention.

FIG. 5 is a diagram showing the basic configuration of the VCO of the FM receiver according to the first embodiment of the present invention.

FIG. 6 is a diagram showing a configuration in which a correction unit is added to the VCO of the FM receiver according to the first embodiment of the present invention.

FIG. 7 is a flowchart illustrating a method for adjusting the FM receiver according to the first embodiment of the present invention.

FIG. 8 is a block diagram showing another configuration of the biosignal measurement system according to the first embodiment of the present invention.

FIG. 9 is a block diagram showing the configuration of a biosignal measurement system according to a second embodiment of the present invention.

FIG. 10 is a block diagram showing another configuration of the biosignal measurement system according to the second embodiment of the present invention.

FIG. 11 is a diagram showing the basic configuration of an FM transmitter according to the second embodiment of the present invention.

FIG. 12 is a diagram showing a configuration in which a correction unit is added to the VCO of the FM transmitter according to the second embodiment of the present invention.

FIG. 13 is a flowchart illustrating a method for adjusting the FM transmitter according to the second embodiment of the present invention.

FIG. 14 is a block diagram showing the configuration of a biosignal measurement system according to a third embodiment of the present invention.

FIG. 15 is a block diagram showing another configuration of the biosignal measurement system according to the third embodiment of the present invention.

FIG. 16 is a flowchart illustrating a method for adjusting an FM transmitter and an FM receiver according to the third embodiment of the present invention.

FIG. 17 is a diagram illustrating the effects of the third embodiment of the present invention.

FIG. 18 is a flowchart illustrating another method for adjusting the FM transmitter and the FM receiver according to the third embodiment of the present invention.

FIG. 19 is a block diagram showing the configuration of a biosignal measurement system according to a fourth embodiment of the present invention.

FIG. 20 is a block diagram showing the configuration of a biosignal measurement system according to a fifth embodiment of the present invention.

FIG. 21 is a block diagram showing an example of the configuration of a computer that implements the biosignal measurement system according to the first to fifth embodiments of the present invention.

FIG. 22 is a diagram showing the configuration of a conventional biosignal measurement system.

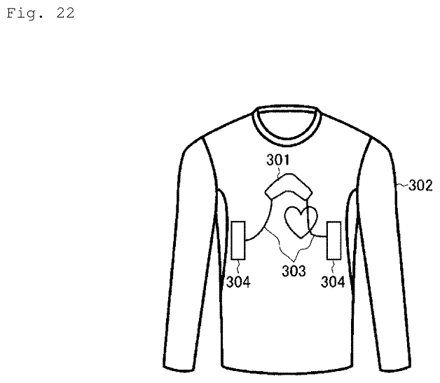

### DETAILED DESCRIPTION OF ILLUSTRATIVE EMBODIMENTS

### First Embodiment

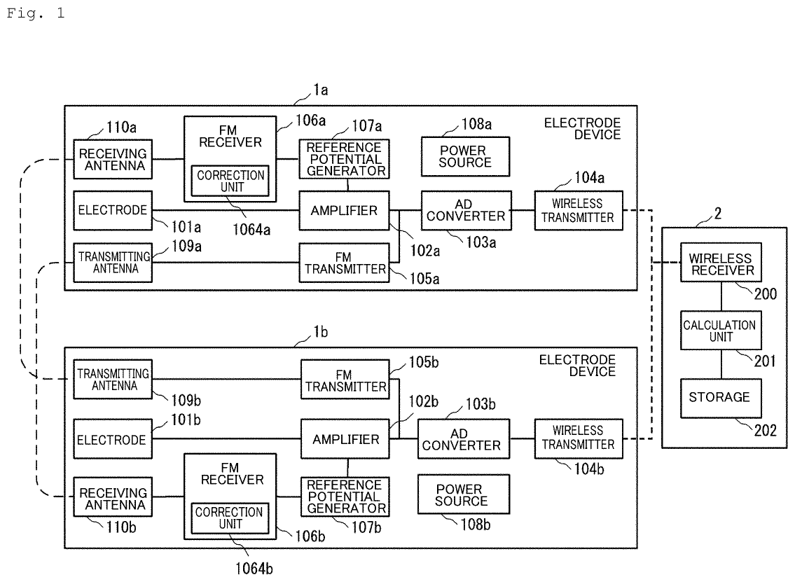

Embodiments of the present invention will be described hereinafter with reference to the drawings. FIG. 1 is a block diagram showing the configuration of a biosignal measurement system according to the present embodiment. The biosignal measurement system includes a sensor device 1a that is attached to a right-side portion of a measurement subject, a sensor device 1b that is attached to a left-side portion of the measurement subject, and a biosignal generation device 2.

The sensor device 1a includes an electrode 101a in contact with the skin on the right-side portion of the measurement subject, an amplifier 102a for amplifying a biopotential detected by the electrode 101a, an AD converter 103a for converting the amplified biopotential into digital data, a wireless transmitter 104a for wirelessly transmitting the digital data output from the AD converter 103a to the biosignal generation device 2, a frequency modulation (FM) transmitter 105a for frequency-modulating a carrier wave according to the biopotential amplified by the amplifier 102a and transmitting the modulated signal to the sensor device 1b, an FM receiver 106a for demodulating the modulated signal transmitted from the sensor device 1b and extracting information on the biopotential, a reference potential generator 107a for generating a reference potential of the amplifier 102a, a power source 108a for supplying power to the amplifier 102a, the AD converter 103a, the wireless transmitter 104a, the FM transmitter 105a, the FM receiver 106a, and the reference potential generator 107a, a transmitting antenna 109a for wirelessly transmitting the modulated signal output from the FM transmitter 105a to the sensor device 1b, and a receiving antenna 110a for receiving the modulated signal transmitted from the sensor device 1b.

The sensor device 1b includes an electrode 101b in contact with the skin on the left-side portion of the measurement subject, an amplifier 102b for amplifying a biopotential detected by the electrode 101b, an AD converter 103b for converting the amplified biopotential into digital data, a wireless transmitter 104b for wirelessly transmitting the digital data output from the AD converter 103b to the biosignal generation device 2, an FM transmitter 105b for frequency-modulating a carrier wave according to the biopotential amplified by the amplifier 102b and transmitting the modulated signal to the sensor device 1a, an FM receiver 106b for demodulating the modulated signal transmitted from the sensor device 1a and extracting information on the biopotential, a reference potential generator 107b for generating a reference potential of the amplifier 102b, a power source 108b for supplying power to the amplifier 102b, the AD converter 103b, the wireless transmitter 104b, the FM transmitter 105b, the FM receiver 106b, and the reference potential generator 107b, a transmitting antenna 109b for wirelessly transmitting the modulated signal output from the FM transmitter 105b to the sensor device 1a, and a receiving antenna 110b for receiving the modulated signal transmitted from the sensor device 1a.

The biosignal generation device 2 is provided with a wireless receiver 200 for receiving the digital data transmitted from the sensor devices 1a and 1b, a calculation unit 201 for calculating an electrocardiogram signal, and a storage 202 for storing the electrocardiogram signal calculated by the calculation unit 201.

When an electrocardiogram signal is measured as a biosignal, it is necessary to arrange a plurality of electrodes 101a and 101b at positions sandwiching the heart of a measurement subject. As a measurement site with a good feeling of use for the measurement subject, the sensor devices 1a and 1b may be attached to at least two locations of the limbs. By adopting such a mounting form of the sensor devices 1a and 1b, the feeling of pressure and discomfort due to wearing of the wear or the like can be greatly reduced. The biosignal measurement system is applicable not only to an electrocardiogram but also to the measurement of myoelectricity, brain waves, and the like.

In the present embodiment, the sensor devices 1a and 1b are in the form of gloves, rings, socks, slippers, or wristbands. The measurement subject wears the sensor devices 1a and 1b by wearing gloves or rings on the right and left hands, respectively. Alternatively, the measurement subject wears the sensor devices 1a and 1b by wearing socks on the right and left feet, respectively, or wearing slippers on the right and left feet, respectively. Alternatively, the measurement subject wears the sensor devices 1a and 1b by putting wristbands on the right and left hands, respectively.

Electrodes of various materials and configurations can be used as the electrodes 101a and 101b. Any electrode such as an Ag/AgCl electrode used in medical applications, a conductive cloth electrode, a metal electrode, or the like can be used. The degree of freedom of the measurement subject can be further improved by forming a non-contact electrode configuration in which the electrode is worn over the clothes by using the electrode made of cloth or metal which does not need to be directly adhered to the body of the measurement subject.

Since the biopotential detected by the electrodes 101a and 101b is a very weak signal, signal amplification by the amplifiers 102a and 102b is required. The amplifiers 102a and 102b require high input impedance to reduce the loss of the biopotential. In the inverting amplifier circuit, the resistance for determining the input impedance also affects the gain setting and further contributes to thermal noise as it is, reducing the SN ratio of the biopotential. On the other hand, the non-inverting amplifier circuit has a feature that noise is less likely to increase even if the circuit has a high input impedance. Therefore, it is effective to use a non-inverting amplifier circuit as the amplifiers 102a and 102b. Further, a low-pass filter may be provided in the amplifiers 102a and 102b.

When a non-inverting amplifier circuit is used as the amplifiers 102a and 102b, it is important that the reference potentials of the two amplifiers 102a and 102b be made common. In the present embodiment, since the sensor devices 1a and 1b are not connected by wiring, the reference potentials of the amplifiers 102a and 102b do not coincide with each other, and the measurement accuracy may deteriorate.

Therefore, in the present embodiment, in order to improve the measurement accuracy of the electrocardiogram, the information of the biopotential is transmitted and received between the sensor devices 1a and 1b, whereby the reference potential Vref of the amplifier 102a of the sensor device 1a and the amplifier 102b of the sensor device 1b is shared.

As will be described later, the biopotential detected by the electrode 101b of the sensor device 1b and amplified by the amplifier 102b is wirelessly transmitted to the sensor device 1a by the FM transmitter 105b. The FM receiver 106a of the sensor device 1a demodulates a signal transmitted from the sensor device 1b and received by the receiving antenna 110a to extract information on the biopotential.

The reference potential generator 107a of the sensor device 1a generates a reference potential Vref by obtaining an addition average of the biopotential detected by the electrode 101a and amplified by the amplifier 102a and the biopotential (biopotential transmitted from the sensor device 1b) output from the FM receiver 106a.

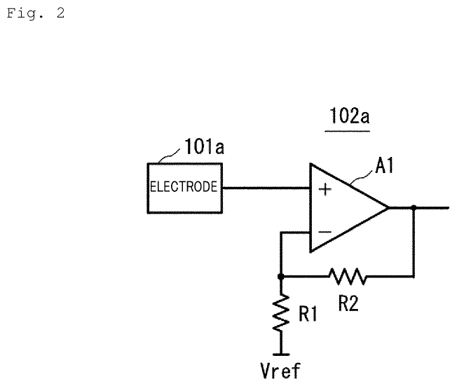

FIG. 2 is a circuit diagram showing an example of the configuration of the amplifier 102a. The amplifier 102a is composed of an operational amplifier A1 and resistors R1 and R2. The reference potential Vref is supplied from the reference potential generator 107a to one end of the resistor R1 of the amplifier 102a.

On the other hand, the biopotential detected by the electrode 101a of the sensor device 1a and amplified by the amplifier 102a is wirelessly transmitted to the sensor device 1b by the FM transmitter 105a. The FM receiver 106b of the sensor device 1b demodulates a signal transmitted from the sensor device 1a and received by the receiving antenna 110b to extract information on the biopotential.

The reference potential generator 107b of the sensor device 1b generates a reference potential Vref by obtaining an addition average of the biopotential detected by the electrode 101b and amplified by the amplifier 102b and the biopotential (biopotential transmitted from the sensor device 1a) output from the FM receiver 106b, and supplies the reference potential Vref to the amplifier 102b. The configuration of the amplifier 102b is the same as that of the amplifier 102a. It is suitable for each of the reference potential generators 107a and 107b to comprise, for example, a single-stage operational amplifier.

The AD converter 103a of the sensor device 1a converts the biopotential amplified by the amplifier 102a into digital data. The wireless transmitter 104a wirelessly transmits the data of the biopotential output from the AD converter 103a to the biosignal generation device 2.

Similarly, the AD converter 103b of the sensor device 1b converts the biopotential amplified by the amplifier 102b into digital data. The wireless transmitter 104b wirelessly transmits the data of the biopotential output from the AD converter 103b to the biosignal generation device 2.

As the wireless communication standard between the wireless transmitters 104a and 104b and the wireless receiver 200 of the biosignal generation device 2, any standard such as carrier communication, Wi-Fi (registered trademark), or Bluetooth (registered trademark) can be applied. When a short-range communication standard such as Bluetooth is adopted, a smartphone or the like which is a terminal familiar to the measurement subject can be used as the biosignal generation device 2. When Wi-Fi or the like is adopted, a server device or the like can be used as the biosignal generation device 2.

The calculation unit 201 of the biosignal generation device 2 calculates a difference between the biopotential transmitted from the sensor device 1a and the biopotential transmitted from the sensor device 1b as an electrocardiogram signal. The electrocardiogram signal is stored in the storage 202.

Next, the FM transmission and reception between the sensor devices 1a and 1b of the present embodiment will be described in more detail. The FM transmitter 105a of the sensor device 1a FM-modulates a carrier wave according to the biopotential amplified by the amplifier 102a, and transmits the modulated signal from the transmitting antenna 109a to the sensor device 1b. The FM receiver 106a of the sensor device 1a demodulates the modulated signal transmitted from the sensor device 1b and received by the receiving antenna 110a to extract information on the biopotential.

Similarly, the FM transmitter 105b of the sensor device 1b FM-modulates a carrier wave according to the biopotential amplified by the amplifier 102b, and transmits the modulated signal from the transmitting antenna 109b to the sensor device 1a. The FM receiver 106b of the sensor device 1b demodulates the modulated signal transmitted from the sensor device 1a and received by the receiving antenna 110b to extract information on the biopotential.

In the present embodiment, a voltage-controlled oscillator (VCO) is used for FM modulation of a carrier wave in the FM transmitters 105a and 105b, and a phase locked loop (PLL) is used for demodulation in the FM receivers 106a and 106b. The VCO generally changes the oscillation frequency by using a varactor diode whose capacitance changes with voltage. The oscillation frequency f of the VCO is expressed by the following equation, and is determined by the resonance frequency of the inductance L and the capacitance C.

$$\begin{matrix}[ Math . 1 ] & \\ f = \frac{1}{2 π \sqrt{LC}} & ( 1 )\end{matrix}$$

The capacitance C is often configured by a varactor diode and a plurality of capacitors. In addition, a VCO is used for components of the PLL of the FM receivers 106a and 106b. Therefore, it is necessary to set the oscillation frequency equal between the VCOs of the FM transmitters 105a and 105b and the FM receivers 106a and 106b.

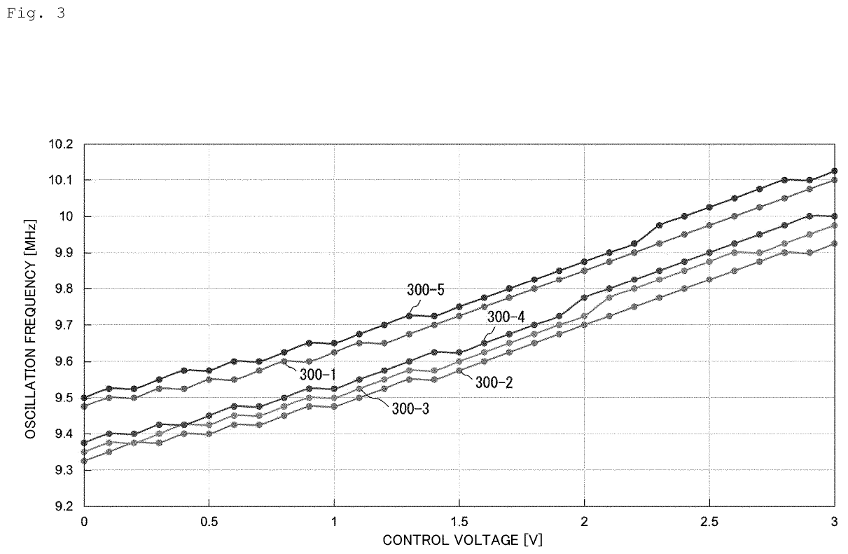

However, since the oscillation frequency varies depending on the accuracy or individual difference of the VCO elements, the oscillation frequency may differ between the transmitting side and the receiving side. FIG. 3 shows an example of individual differences in oscillation frequencies of the VCO. Here, the control voltage-oscillation frequency characteristics of five VCOs 300-1 to 300-5 are shown. All VCOs use the same element in design, and theoretically have to exhibit the same control voltage-oscillation frequency characteristics, but a large individual difference occurs.

In the example shown in FIG. 3, an offset error of about 200 kHz is generated between the VCOs 300-2 and 300-5, and an error of about 1 V is obtained when converted into a voltage. Considering that the circuit voltage used in a wearable device, or the like, is 3 V to 5 V, this error corresponds to 20% or more of the circuit voltage, and extremely greatly affects the measurement result.

In the present embodiment, the biopotentials detected by the sensor devices 1a and 1b are mutually transmitted and received, so that the reference potential Vref of the amplifier 102a and the amplifier 102b are made to coincide with each other, and the measurement accuracy of the electrocardiogram signal is improved. However, when the biopotentials detected by the sensor devices 1a and 1b cannot be correctly demodulated, the measurement accuracy of the electrocardiogram signal remarkably deteriorates, so that it is very important to correct the error of the oscillation frequency of the VCO.

As one example of the correction method, it is conceivable to add an offset to the reference potentials Vref generated by the reference potential generators 107a and 107b, or adjust the amplification degrees of the reference potential generators 107a and 107b to adjust the inclination of the control voltage-oscillation frequency of the VCO. However, although the first-order correction function is used in this correction method, since two variables of the slope of the VCO control voltage-oscillation frequency and the intercept have to be solved, the process becomes complicated. Further, in order to make the inclination of the control voltage-oscillation frequency of the VCO less than 1, for example, an inverting amplifier circuit is required, but in order to invert the output, the noise gain becomes larger than the signal gain, and the noise increases.

Further, when a variable resistor is used to adjust the amplification degree of the reference potential generators 107a and 107b, there is a possibility that the correction is shifted over time due to drift caused by time or temperature changes. When the difference in the oscillation frequency of the VCO is large, the control voltage corresponding to the oscillation frequency may be saturated at the value of the power supply voltage (GND or VDD), so that information for calculating the first-order correction function cannot be obtained, and correction may not be performed.

Therefore, the solution is to correct the oscillation frequency itself of the VCO. In addition, according to FIG. 3, although a large individual difference exists in the VCO, the change rate of the oscillation frequency with respect to the control voltage has little individual difference compared to the offset of the oscillation frequency, and the inclination of the control voltage-oscillation frequency is almost constant.

Therefore, in the present embodiment, correction is performed by changing the offset of the oscillation frequency. More specifically, the VCOs of the FM receivers 106a and 106b of the sensor devices 1a and 1b are provided with correction units 1064a and 1064b, respectively.

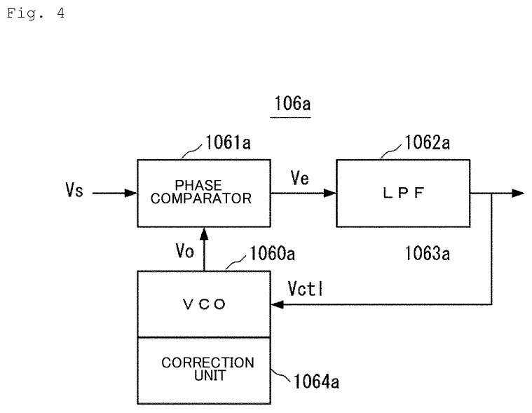

FIG. 4 is a diagram showing the basic configuration of the FM receiver 106a. As described above, the FM receiver 106a has a PLL configuration and includes a VCO 1060a, a phase comparator 1061a for phase-comparing the modulated signal Vs received by the receiving antenna 110a with the output signal Vo of the VCO 1060a, a low-pass filter (LPF) 1062a for outputting the result of low-pass filtering the output signal Ve of the phase comparator 1061a as a demodulated biopotential, and a correction unit 1064a. The output of the LPF 1062a is input to the VCO 1060a as a control voltage Vctl. The configuration of the FM receiver 106b is the same as that of the FM receiver 106a.

The configuration shown in FIG. 4 is an example, and the FM receivers 106a and 106b are not limited to the configuration shown in FIG. 4. For example, an amplifier may be inserted between the LPF 1062a and the VCO 1060a, and a voltage obtained by amplifying the biopotential output from the LPF 1062a may be used as a control voltage Vctl of the VCO 1060a.

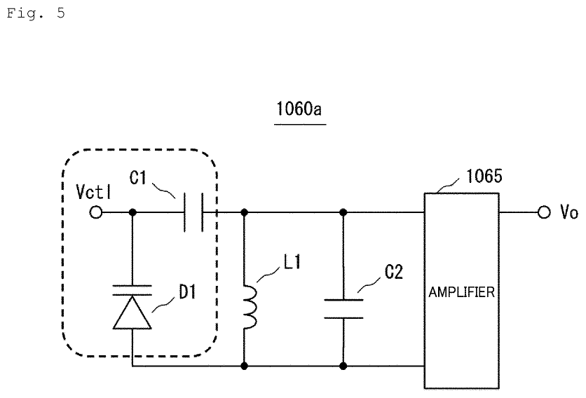

FIG. 5 is a diagram showing the basic configuration of the VCO 1060a. The VCO 1060a includes a varactor diode D1, capacitors C1 and C2, an inductor L1, and an amplifier 1065. When the fluctuation component of capacitance by the varactor diode D1 and the capacitor C1 is Cᵥₐᵣ, the constant component of capacitance by the capacitor C2 is C_{const}, and the inductance of the inductor L1 is L, the oscillation frequency f of the VCO 1060a is expressed as follows.

$$\begin{matrix}[ Math . 2 ] & \\ f = \frac{1}{2 π \sqrt{L ( {C}_{var} + {C}_{const} )}} & ( 2 )\end{matrix}$$

When the inductance L is changed, the coefficient of the fluctuation component is also changed, so that it is difficult to obtain a desired frequency characteristic. Therefore, it is efficient to change the oscillation frequency f by changing the capacitance C_{const}.

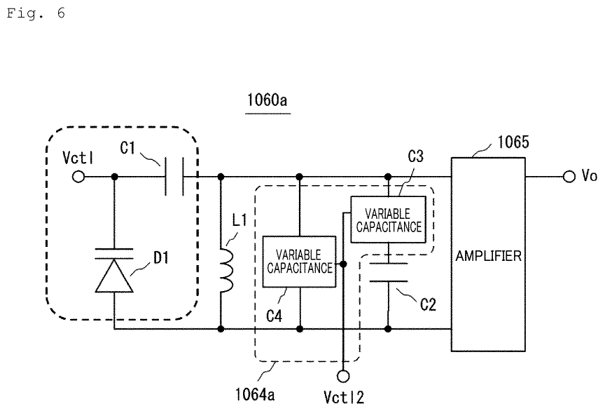

FIG. 6 is a diagram showing a configuration in which the correction unit 1064a is added to the VCO 1060a. The oscillation frequency f of the VCO 1060a decreases when the capacitance is increased and increases when the capacitance is decreased. Therefore, the offset of the oscillation frequency f of the VCO 1060a can be changed by providing variable capacitors C3 and C4 in series and in parallel with the capacitor C1 as the correction unit 1064a.

The variable capacitors C3 and C4 can be configured of a varactor diode, a MEMS (Micro Electro Mechanical Systems) variable capacitor, and a capacitor arranged in an array. In a circuit configuration using discrete components, implementation using a varactor diode or MEMS variable capacitor is advantageous because it requires fewer components and is easier to control. On the other hand, when the circuit is integrated into an LSI (Large Scale Integration), it is preferable to use a capacitor array as the variable capacitors C3 and C4 because it is easier to implement. The configuration of the correction unit 1064b is the same as that of the correction unit 1064a.

The configurations of the FM receivers 106a and 106b and the correction units 1064a and 1064b are not limited to the configurations shown in FIGS. 4 to 6, and it is needless to say that a circuit configuration capable of performing the same function may be used.

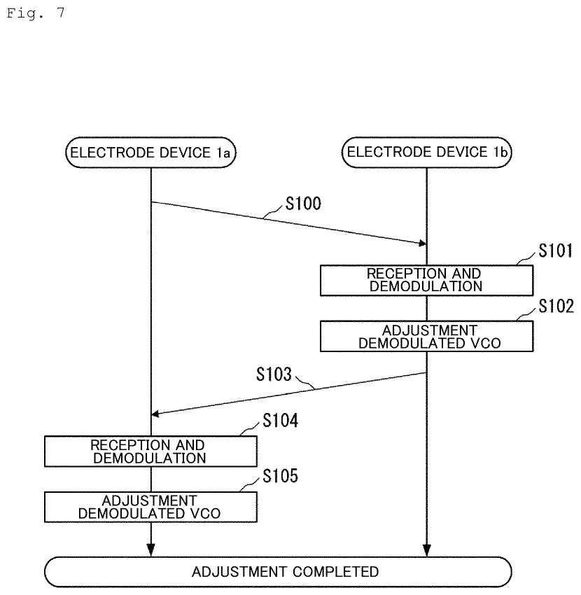

FIG. 7 is a flowchart illustrating a method of adjusting the FM receivers 106a and 106b (correction units 1064a and 1064b). First, an operator who tries to adjust the FM receivers 106a and 106b applies a predetermined arbitrary voltage x[V] to the FM transmitter 105a of the sensor device 1a, and FM-modulates a carrier wave according to the voltage x[V], and transmits the modulated signal from the transmitting antenna 109a to the sensor device 1b (step S100 in FIG. 7).

The FM receiver 106b of the sensor device 1b demodulates the modulated signal transmitted from the sensor device 1a and received by the receiving antenna 110b to output a voltage y[V] (step S101 in FIG. 7). The voltage y[V] is different from the original voltage x[V] due to the error of the VCO of the FM receiver 106b. Then, the operator adjusts the value of the variable capacitance of the correction unit 1064b so that the output voltage y[V] of the VCO coincides with x[V] (step S102 in FIG. 7). The value of the variable capacitance can be set by the control voltage Vctl2.

Then, the operator applies a voltage x[V] to the FM transmitter 105b of the sensor device 1b, FM-modulates the carrier wave according to the voltage x[V], and transmits the modulated signal from the transmitting antenna 109b to the sensor device 1a (step S103 in FIG. 7).

The FM receiver 106a of the sensor device 1a demodulates the modulated signal transmitted from the sensor device 1b and received by the receiving antenna 110a to output a voltage y[V] (step S104 in FIG. 7). The operator adjusts the values of the variable capacitors C3 and C4 of the correction unit 1064a so that the output voltage y[V] of the VCO of the FM receiver 106a coincides with x[V] (step S105 in FIG. 7).

Thus, the adjustment of the FM receivers 106a and 106b (correction units 1064a and 1064b) is completed and the oscillation frequency of the VCO can be corrected, so that the measurement of the biosignal can be started. In the example of FIG. 7, the FM receiver 106a of the sensor device 1a is adjusted after the FM receiver 106b of the sensor device 1b is adjusted first, but the order may be reversed or adjusted simultaneously.

Especially, when the adjustment is performed simultaneously, it is preferable to make the modulated signal transmitted from the sensor device 1a to the sensor device 1b different in frequency from the modulated signal transmitted from the sensor device 1b to the sensor device 1a. The reason for this is that when the same frequency is used, there is a high possibility that the signals interfere with each other. By increasing the frequency to be used, the present disclosure can use more sensor devices as well as a configuration in which sensor devices are paired.

As in the present embodiment, the configuration in which the FM receivers 106a and 106b are provided with the correction units 1064a and 1064b is particularly suitable when human-body communication using the body of the measurement subject as a transmission line is performed. In the case of radio communication in general, where radio waves are transmitted in the air, there is a possibility of erroneous correction in accordance with unnecessary modulated signals scattered in a space. In addition, the allowed radio wave intensity differs depending on the frequency band. Therefore, there is a possibility that the signal may be corrected by entering the frequency band of the strict intensity limit due to the error of the oscillation frequency.

On the other hand, in the case of human-body communication, the signal is rarely affected by the external environment. Since field emission to the external environment is reduced, it is not necessary to tune the transmission frequency to a specific frequency unlike radio wave communication. Moreover, since it is only necessary to tune the oscillation frequency of the VCO of each of the FM receivers 106a and 106b to the transmission frequency of the sensor devices 1a and 1b, a correction function can be realized with a simple mechanism.

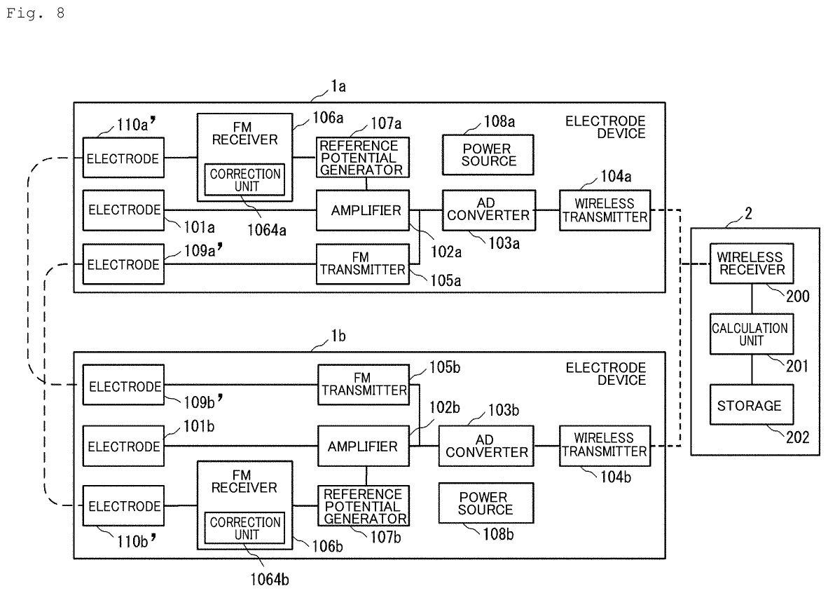

FIG. 8 shows the configuration of a biosignal measurement system in the case of using human-body communication. In the configuration shown in FIG. 8, electrodes 109a′ and 110a′ being in contact with the skin on the right-side portion of the measurement subject are provided instead of the antennas 109a and 110a shown in FIG. 1. Electrodes 109b′ and 110b′ being in contact with the skin on the left-side portion of the measurement subject are provided instead of the antennas 109b and 110b.

In the configuration shown in FIG. 8, the FM transmitter 105a of the sensor device 1a FM-modulates a carrier wave according to the biopotential amplified by the amplifier 102a, and transmits the modulated signal from the electrode 109a′ to the sensor device 1b via the body of the measurement subject. The FM receiver 106a demodulates the modulated signal transmitted from the sensor device 1b and received by the electrode 110a′ to extract information on the biopotential.

Similarly, the FM transmitter 105b of the sensor device 1b FM-modulates a carrier wave according to the biopotential amplified by the amplifier 102b, and transmits the modulated signal from the electrode 109b′ to the sensor device 1a via the body of the measurement subject. The FM receiver 106b demodulates the modulated signal transmitted from the sensor device 1a and received by the electrode 110b′ to extract information on the biopotential.

The power for communication accounts for most of the power consumption of the sensor devices 1a and 1b. In spatial propagation using radio waves, the signal intensity is attenuated inversely proportional to the square of the propagation distance. On the other hand, in the case of human-body communication, the signal attenuates in inverse proportion to the propagation distance. Thus, human-body communication enables data transmission with less transmission power. The transmission/reception of data is performed through the human body, contributing to the reduction of power consumption.

As described above, in the present embodiment, since the oscillation frequencies of the VCOs of the FM receivers 106a and 106b can be corrected, the variation of the oscillation frequencies of the FM transmitters 105a and 105b and the FM receivers 106a and 106b caused by the influence from the external environment can be minimized. Moreover, since the reference potentials for measuring the potentials of the sensor devices 1a and 1b can be made common, the measurement accuracy of the biopotential can be improved. In the present embodiment, it is not necessary to specify the accurate oscillation frequencies of the FM transmitter of one sensor device and the FM receiver of the other sensor device, and it is sufficient that the oscillation frequencies of the FM transmitter and the FM receiver can be tuned, so that the correction can be realized at a low cost with a simple circuit.

### Second Embodiment

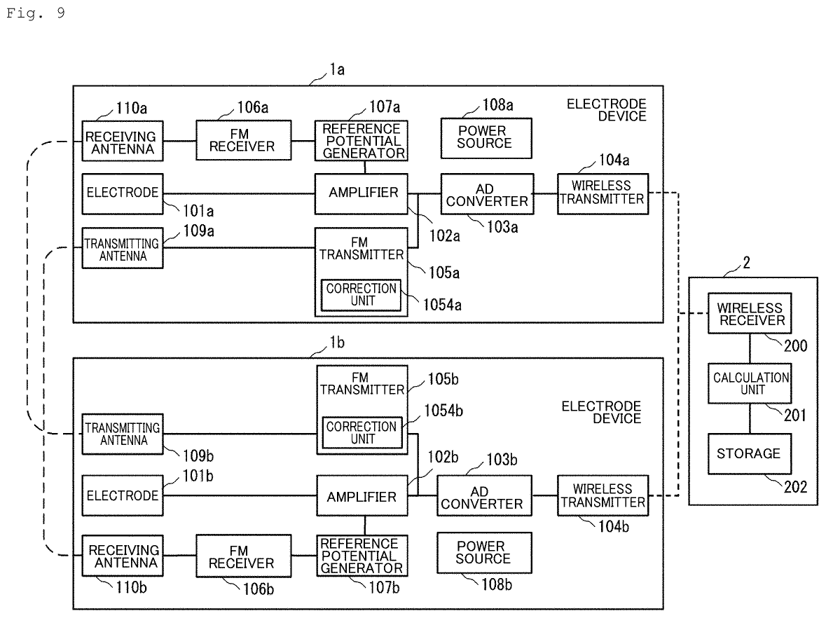

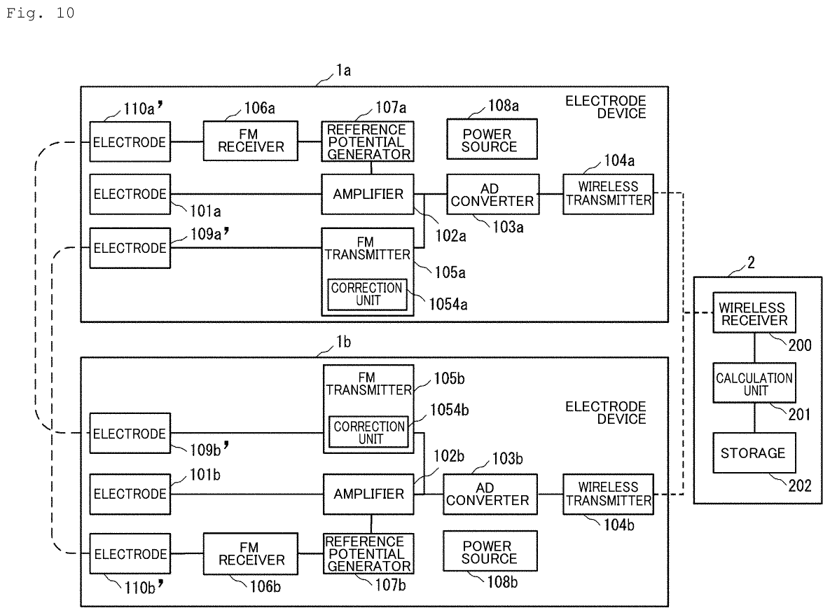

In the configuration shown in FIGS. 1 and 8, the VCOs of the FM receivers 106a and 106b of the sensor devices 1a and 1b are provided with the correction units 1064a and 1064b, but the VCOs of the FM transmitters 105a and 105b may be provided with correction units. FIG. 9 shows a configuration in which the VCOs of the FM transmitters 105a and 105b in the configuration of FIG. 1 are provided with correction units 1054a and 1054b, and FIG. 10 shows a configuration in which the VCOs of the FM transmitters 105a and 105b in the configuration of FIG. 8 are provided with the correction units 1054a and 1054b.

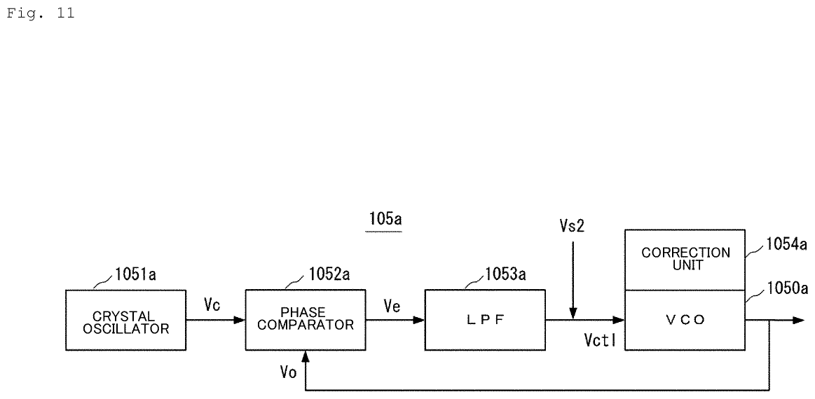

FIG. 11 is a diagram showing the basic configuration of the FM transmitter 105a in the configurations of FIGS. 9 and 10. The FM transmitter 105a includes a VCO 1050a, a crystal oscillator 1051a for outputting a carrier wave Vc, a phase comparator 1052a for phase-comparing the carrier wave Vc with an output signal Vo of the VCO 1050a, an LPF 1053a for outputting a result of low-pass filtering the output signal Ve of the phase comparator 1052a, and a correction unit 1054a. A signal obtained by mixing an output signal of the LPF 1053a with the biopotential Vs2 amplified by the amplifier 102a is input to the VCO 1050a as a control voltage Vctl. An output signal Vo of the VCO 1050a is output to the transmitting antenna 109a or the electrode 109a′ as a modulated signal. The configuration of the FM transmitter 105b is the same as that of the FM transmitter 105a.

The configuration of FIG. 11 is one example, and the FM transmitters 105a and 105b are not limited to the configuration of FIG. 11. For example, an amplifier may be inserted between the VCO 1050a and the transmitting antenna 109a or the electrode 109a′ to amplify the modulated signal. Further, a frequency divider may be inserted between the crystal oscillator 1051a and the phase comparator 1052a and between the VCO 1050a and the phase comparator 1052a, respectively.

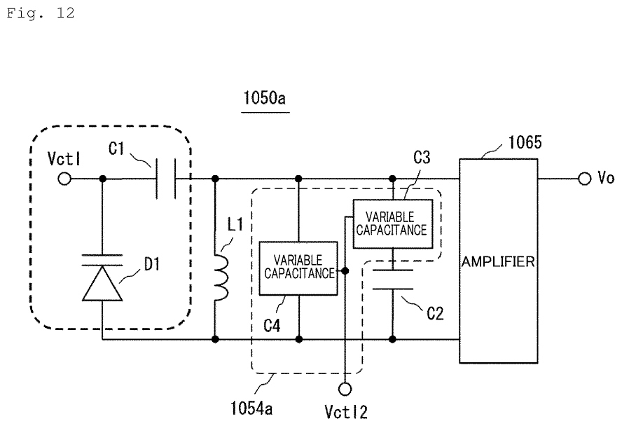

FIG. 12 is a diagram showing a configuration in which the correction unit 1054a is added to the VCO 1050a. Since the configuration of the VCO 1050a is the same as that of the VCO 1060a and the configuration of the correction unit 1054a is the same as that of the correction unit 1064a, the description thereof will be omitted. The configuration of the correction unit 1054b is the same as that of the correction unit 1054a.

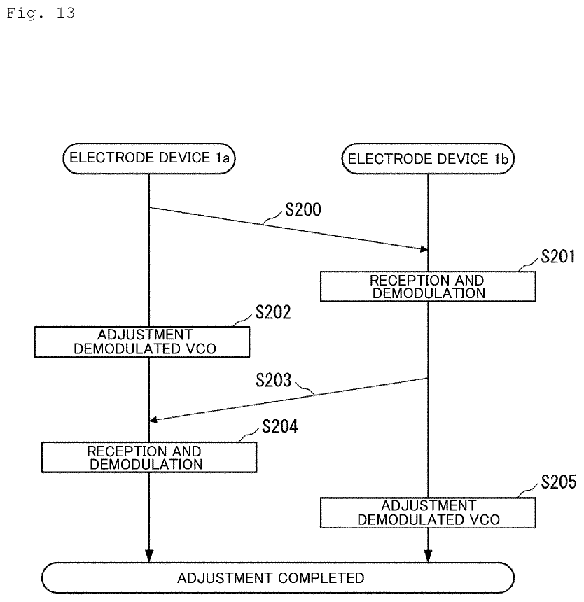

FIG. 13 is a flowchart illustrating a method of adjusting the FM transmitters 105a and 105b (correction units 1054a and 1054b) of the present embodiment. First, an operator who tries to adjust the FM transmitters 105a and 105b applies a predetermined arbitrary voltage x [V] to the FM transmitter 105a of the sensor device 1a, and FM-modulates a carrier wave according to the voltage x[V], and transmits the modulated signal from the transmitting antenna 109a or the electrode 109a′ to the sensor device 1b (step S200 in FIG. 13).

The FM receiver 106b of the sensor device 1b demodulates the modulated signal transmitted from the sensor device 1a and received by the receiving antenna 110b or the electrode 110b′ to output a voltage y[V] (step S201 in FIG. 13). The operator adjusts the values of variable capacitors C3 and C4 of the correction unit 1054a of the FM transmitter 105a so that the output voltage y[V] of the FM receiver 106b coincides with x[V] (step S202 in FIG. 13). The value of the variable capacitance can be set by the control voltage Vctl2.

Then, the operator applies a voltage x[V] to the FM transmitter 105b of the sensor device 1b, FM-modulates the carrier wave according to the voltage x[V], and transmits the modulated signal from the transmitting antenna 109b or the electrode 109b′ to the sensor device 1a (step S203 in FIG. 13).

The FM receiver 106a of the sensor device 1a demodulates the modulated signal transmitted from the sensor device 1b and received by the receiving antenna 110a or the electrode 110a′ to output a voltage y[V] (step S204 in FIG. 13). The operator adjusts the value of the variable capacitance of the correction unit 1054b of the FM transmitter 105b so that the output voltage y[V] of the FM receiver 106a coincides with x[V] (step S205 in FIG. 13).

Thus, the adjustment of the FM transmitters 105a and 105b (correction units 1054a and 1054b) is completed and the oscillation frequency of the VCO can be corrected, so that the measurement of the biosignal can be started. In the example shown in FIG. 13, the FM transmitter 105b of the sensor device 1b is adjusted after the FM transmitter 105a of the sensor device 1a is adjusted first, but the order may be reversed or simultaneously adjusted. As described in the first embodiment, it is preferable that the modulated signal transmitted from the sensor device 1a to the sensor device 1b and the modulated signal transmitted from the sensor device 1b to the sensor device 1a have different frequencies.

### Third Embodiment

In the first and second embodiments, either the FM transmitter or the FM receiver is provided with a correction unit, but both the FM transmitter and the FM receiver may be provided with a correction unit.

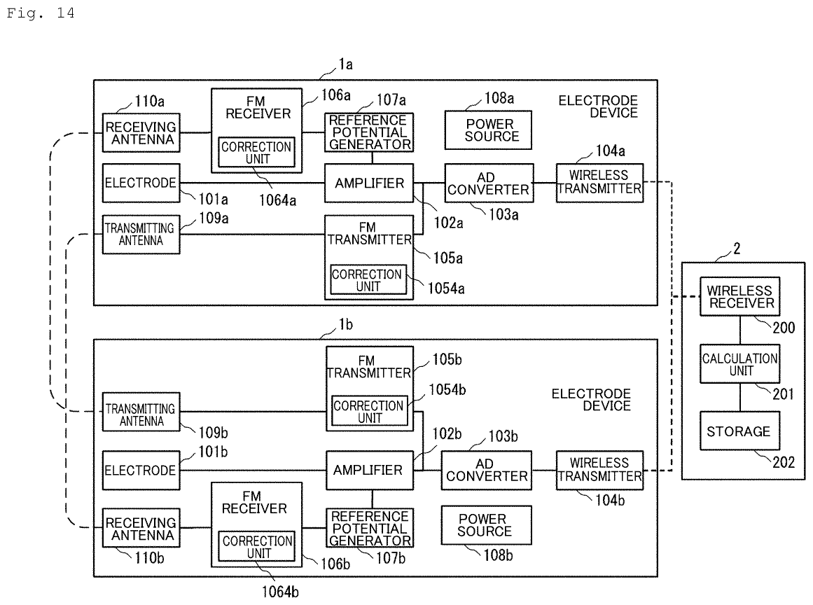

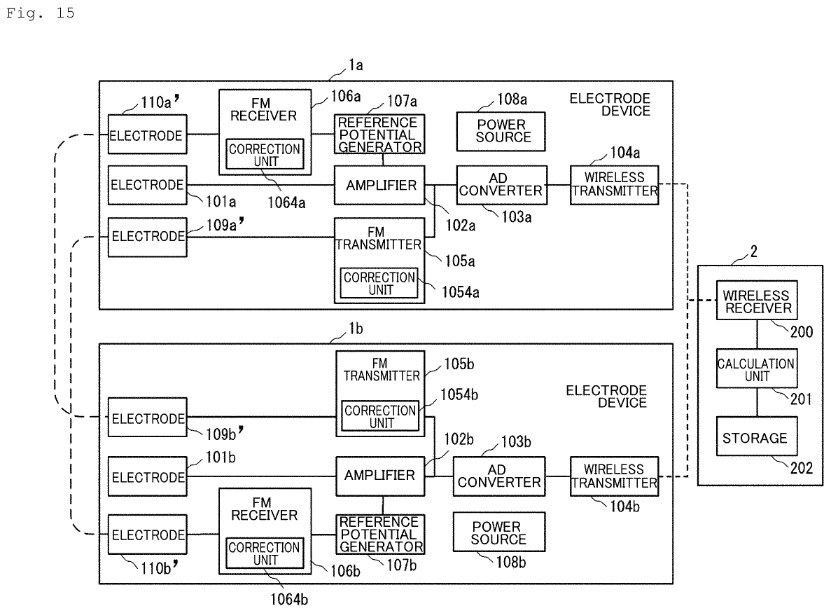

FIG. 14 is a block diagram showing the configuration of the biosignal measurement system according to the present embodiment. The configuration of the FM receivers 106a and 106b is as described in the first embodiment, and the configuration of the FM transmitters 105a and 105b is as described in the second embodiment. FIG. 15 shows the configuration of a biosignal measurement system in the case of using human-body communication.

In the present embodiment, the correction amount is increased when the correction is not completed by the methods of the first and second embodiments. There is a possibility that a sufficient correction amount cannot be obtained due to the variation range of the variable capacitance of the correction units 1054a, 1054b, 1064a, and 1064b and the variation of the modulator/demodulator. Then, in the present embodiment, the correction units 1054a and 1054b are added to the FM transmitters 105a and 105b in addition to the correction units 1064a and 1064b of the FM receivers 106a and 106b of the present embodiment.

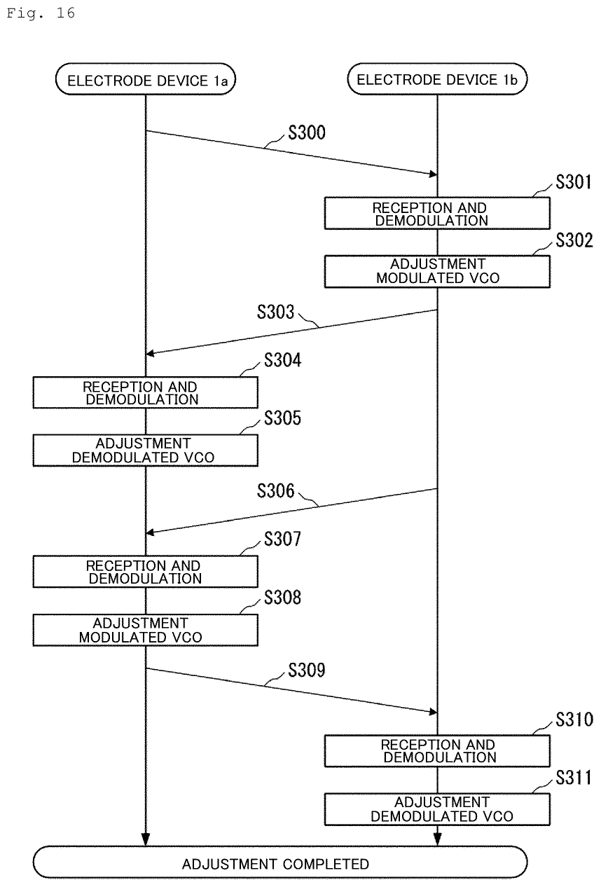

FIG. 16 is a flowchart for explaining the adjustment method of the FM transmitters 105a and 105b and the FM receivers 106a and 106b (correction units 1054a, 1054b, 1064a, and 1064b). In this case, a flowchart is shown when the correction of the sensor device 1b is not sufficient.

First, an operator applies a predetermined arbitrary voltage x1[V] to the FM transmitter 105a of the sensor device 1a, FM-modulates a carrier wave according to the voltage x1[V], and transmits the modulated signal from the transmitting antenna 109a or the electrode 109a′ to the sensor device 1b (step S300 in FIG. 16).

The FM receiver 106b of the sensor device 1b demodulates the modulated signal transmitted from the sensor device 1a and received by the receiving antenna 110b or the electrode 110b′ to output a voltage y1 [V] (step S301 in FIG. 16). The operator adjusts the value of the variable capacitance of the correction unit 1064b so that the output voltage y1 [V] of the FM receiver 106b coincides with x1[V] (step S302 in FIG. 16).

Then, the operator applies a predetermined arbitrary voltage x2 [V] to the FM transmitter 105b of the sensor device 1b, FM-modulates a carrier wave according to the voltage x2 [V], and transmits the modulated signal from the transmitting antenna 109b or the electrode 109b′ to the sensor device 1a (step S303 in FIG. 16).

The FM receiver 106a of the sensor device 1a demodulates the modulated signal transmitted from the sensor device 1b and received by the receiving antenna 110a or the electrode 110a′ to output a voltage y2 [V] (step S304 in FIG. 16). The operator adjusts the values of the variable capacitors C3 and C4 of the correction unit 1064a so that the output voltage y2 [V] of the FM receiver 106a coincides with x2 [V] (step S305 in FIG. 16). As described in the first embodiment, the adjustment of steps S300 to S302 and the adjustment of steps S303 to S305 may be performed simultaneously.

Here, it is assumed that the output voltage y1 [V] and x1[V] of the FM receiver 106b do not coincide with each other even if the value of the variable capacitance of the correction unit 1064b is adjusted, and the correction is not completed. An operator applies a predetermined arbitrary voltage a2 [V] to the FM transmitter 105b of the sensor device 1b whose correction has not been completed, FM-modulates a carrier wave according to the voltage a2 [V], and transmits the modulated signal from the transmitting antenna 109b or the electrode 109b′ to the sensor device 1a (step S306 in FIG. 16).

The FM receiver 106a of the sensor device 1a demodulates the modulated signal transmitted from the sensor device 1b and received by the receiving antenna 110a or the electrode 110a′ and outputs a voltage β2 [V] (step S307 in FIG. 16).

When the output voltage β2 [V] of the FM receiver 106a is equal to or higher than a fixed threshold voltage Vth [V], an operator raises the oscillation frequency of the VCO 1050a of the FM transmitter 105a, that is, decreases the values of the variable capacitors C3 and C4 of the correction unit 1054a. When the output voltage β2 [V] of the FM receiver 106a is less than the threshold voltage Vth [V], the operator lowers the oscillation frequency of the VCO 1050a, that is, increases the variable capacitors C3 and C4 of the correction unit 1054a (step S308 in FIG. 16). The threshold voltage Vth may be, for example, Vdd/2 (Vdd is a power supply voltage).

The correction amount of the correction unit 1054a at this time may be a selectable maximum amount. When all the correction amounts of transmission and reception are made equal, the correction amount of the correction unit 1054a may be changed by the maximum amount, so that a number of repeated adjustment operations are not required, and the adjustment operations can be easily performed. The processing in steps S309 to S311 in FIG. 16 is the same as steps S300 to S302.

Thus, the adjustment of the FM transmitters 105a and 105b and the FM receivers 106a and 106b (correction units 1054a, 1054b, 1064a, and 1064b) is completed and the oscillation frequency of the VCO can be corrected, so that the measurement of the biosignal can be started.

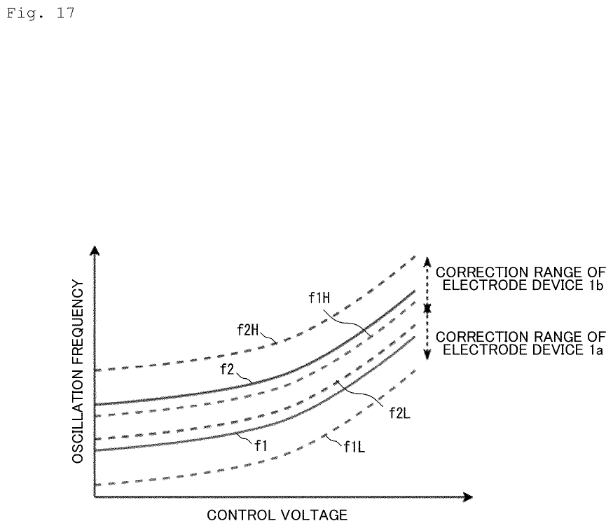

It is assumed that the control voltage-oscillation frequency characteristics of the VCO of the FM receiver 106b of the sensor device 1b are in the range of f2H to f2L shown in FIG. 17, and the control voltage-oscillation frequency characteristics of the VCO of the FM transmitter 105a of the sensor device 1a are in the range of f1H to fiL. In this case, even if the oscillation frequency f2 of the VCO of the FM receiver 106b is adjusted to the lower limit value f2L by the correction unit 1064b, the lower limit value f2L does not reach the oscillation frequency f1 of the VCO of the FM transmitter 105a of the sensor device 1a.

However, when the oscillation frequency f1 of the VCO of the FM transmitter 105a is adjusted to the upper limit value f1H by the correction unit 1054a, the upper limit value f1H is within the correction range of the sensor device 1b, so that complete correction is made possible. Even if the magnitude relationship of the frequencies is different, the correction is performed by the same discussion.

According to the present embodiment, the correction amounts in both the FM transmitter and the FM receiver can be reduced by providing the correction units in both the FM transmitter and the FM receiver. If the capacitance Cconst described in Equation (2) is largely corrected, the ratio of the capacitance Cconst to Cvar is lost, and there is a possibility that the inclination of the control voltage-oscillation frequency characteristics of the VCO is deviated between the FM transmitter and the FM receiver. According to the present embodiment, such inclination deviation can be suppressed.

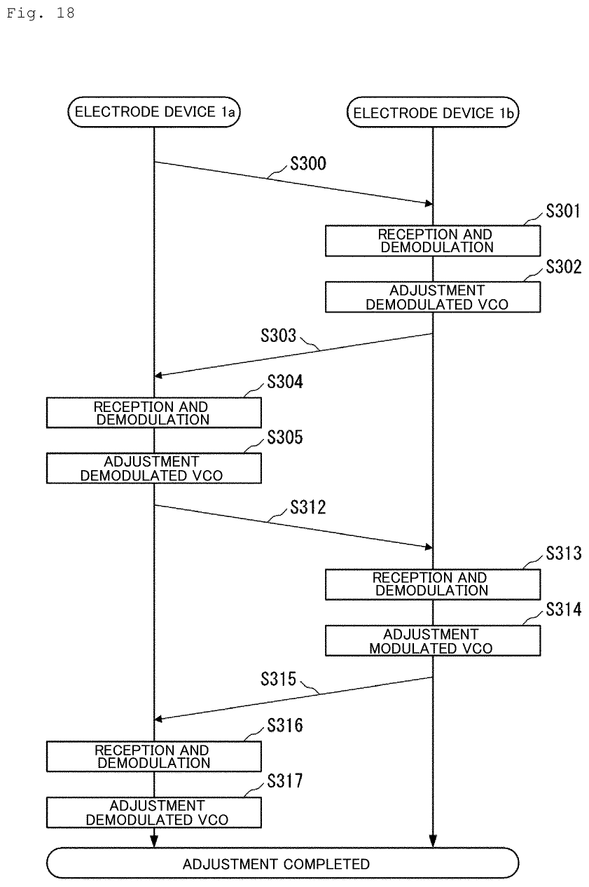

Even if the value of the variable capacitance of the correction unit 1064a is adjusted, when the output voltages y2 [V] and x2 [V] of the FM receiver 106a do not coincide with each other and correction is not completed, the adjustment described in FIG. 18 may be performed. The processing in steps S300 to S305 in FIG. 18 is the same as described in FIG. 16.

An operator applies a predetermined arbitrary voltage α1 [V] to the FM transmitter 105a of the sensor device 1a whose correction has not been completed, FM-modulates a carrier wave according to the voltage α1 [V], and transmits the modulated signal from the transmitting antenna 109a or the electrode 109a′ to the sensor device 1b (step S312 in FIG. 18).

The FM receiver 106b of the sensor device 1b demodulates the modulated signal transmitted from the sensor device 1a and received by the receiving antenna 110b or the electrode 110b′ to output a voltage β1 [V] (step S313 in FIG. 18).

When the output voltage β1 [V] of the FM receiver 106b is equal to or higher than the threshold voltage Vth [V], an operator raises the oscillation frequency of the VCO of the FM transmitter 105b, that is, decreases the value of the variable capacitance of the correction unit 1054b. When the output voltage β1 [V] of the FM receiver 106b is less than the threshold voltage Vth [V], the operator lowers the oscillation frequency of the VCO of the FM transmitter 105b, that is, increases the value of the variable capacitance of the correction unit 1054b (step S314 in FIG. 18). The processing in steps S315 to S317 in FIG. 18 is the same as steps S303 to S305.

### Fourth Embodiment

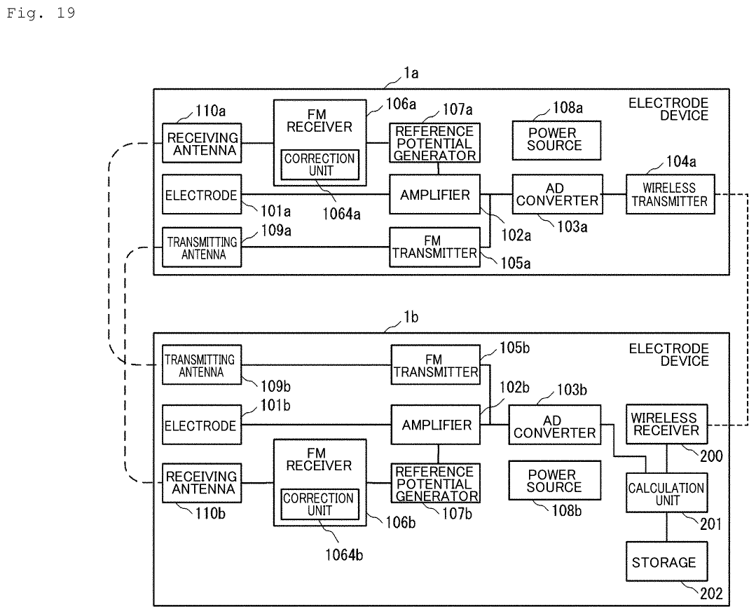

In the first to third embodiments, the biosignal generation device 2 is provided separately from the sensor devices 1a and 1b, but the configuration of the biosignal generation device 2 may be mounted on either of the sensor devices 1a and 1b. FIG. 19 is a block diagram showing the configuration of the biosignal measurement system of the present embodiment.

In the configuration shown in FIG. 19, the wireless transmitter 104b of the sensor device 1b is not necessary. The wireless receiver 200 provided in the sensor device 1b receives data of the biopotential transmitted from the sensor device 1a. The calculation unit 201 calculates a difference between the biopotential transmitted from the sensor device 1a and the biopotential output from the AD converter 103b as an electrocardiogram signal. The electrocardiogram signal is stored in the storage 202.

In I the present embodiment, since it is not necessary to provide the biosignal generation device 2 separately from the sensor devices 1a and 1b, it is not necessary for the measurement subject to carry the biosignal generation device 2, and the convenience of the measurement subject can be improved.

In the example shown in FIG. 19, the sensor device 1b is provided with the configuration of the biosignal generation device 2, but it is needless to say that the sensor device 1a may be provided.

Further, although the configuration of FIG. 19 shows an example in which the present embodiment is applied to the configuration of FIG. 1, the present embodiment may be applied to the configurations of FIGS. 8 to 10, 14, and 15.

### Fifth Embodiment

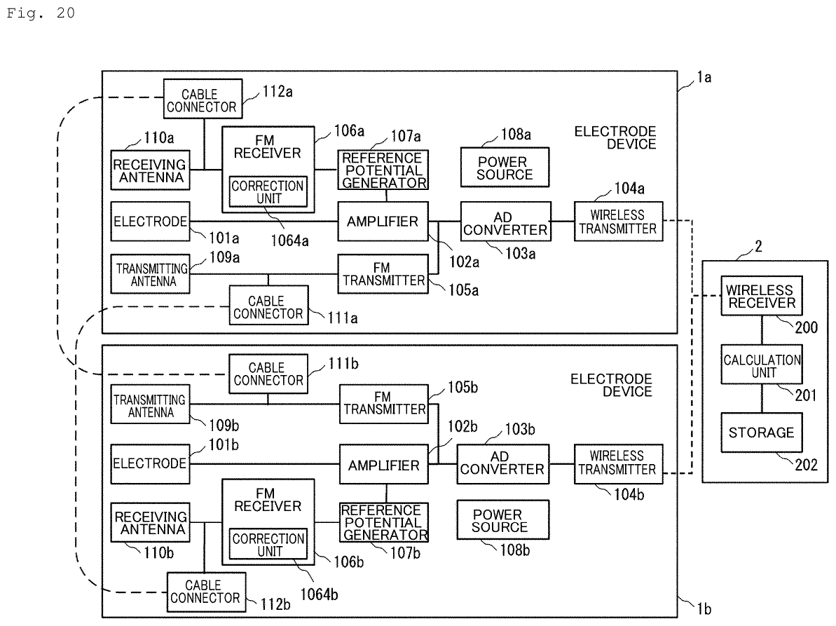

In the first to fourth embodiments, adjustment is performed by wireless communication even at the time of adjustment before starting measurement of the biosignal, but the adjustment may be performed by wired communication by providing a cable connector in the sensor devices 1a and 1b. FIG. 20 is a block diagram showing the configuration of the biosignal measurement system of the present embodiment.

The sensor device 1a includes an electrode 101a, an amplifier 102a, an AD converter 103a, a wireless transmitter 104a, an FM transmitter 105a, an FM receiver 106a, a reference potential generator 107a, a power source 108a, a transmitting antenna 109a, a receiving antenna 110a, and cable connectors 111a and 112a for performing wired communication with the sensor device 1b.

The sensor device 1b includes an electrode 101b, an amplifier 102b, an AD converter 103b, a wireless transmitter 104b, an FM transmitter 105b, an FM receiver 106b, a reference potential generator 107b, a power source 108b, a transmitting antenna 109b, a receiving antenna 110b, and cable connectors 111b and 112b for performing wired communication with the sensor device 1a.

According to the present embodiment, for example, the sensor devices 1a and 1b can be connected by wiring in a case for storing a pair of sensor devices 1a and 1b, and adjustment can be performed. The power sources 108a and 108b of the sensor devices 1a and 1b are batteries, and a device for charging the batteries is required. When the sensor devices 1a and 1b are mounted on a charging case, the cable connectors 111a and 112b are connected by wiring in the charging case simultaneously with the charging of the battery, and the cable connectors 111b and 112a are connected.

The adjustment method is different from the first to fourth embodiments in that wireless communication in steps S100 and S103 of FIG. 7, steps S200 and S203 of FIG. 13, steps S300, S303, S306, and S309 of FIG. 16, and steps S300, S303, S312, and S315 of FIG. 18 is changed to wired communication.

The configuration in FIG. 20 shows an example in which the present embodiment is applied to the configuration in FIG. 1, but the present embodiment may also be applied to the configurations in FIGS. 8 to 10, 14, 15, and 19. The sensor devices 1a and 1b may be connected by wiring without interposing a charging case.

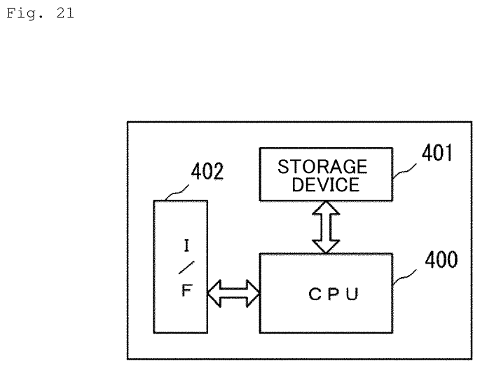

The calculation unit 201 and storage 202 described in the first to fifth embodiments can be realized by a computer equipped with a CPU (Central Processing Unit), a storage device, and an interface, and a program that controls these hardware resources. FIG. 21 illustrates a configuration example of the computer.

The computer includes a CPU 400, a storage device 401, and an interface device (I/F) 402. The hardware of the wireless receiver 200 and the like are connected to the I/F 402. A program for implementing the method of the present disclosure is stored in the storage device 401. The CPU 400 executes the processes described in the first to fifth embodiments according to the program stored in the storage device 401. Furthermore, at least a portion of the calculation unit 201 may be configured with hardware logic such as a field-programmable gate array (FPGA).

Some or all of the examples are also described in the following supplementary notes, but are not limited to the following.
(Supplementary note 1) A biosignal measurement system including:
a first sensor device configured to be attached to one of a right-side portion and a left-side portion of a measurement subject; and
a second sensor device configured to be attached to the other of the right-side portion and the left-side portion, wherein
each of the first sensor device and the second sensor device includes:
a first electrode configured to be in contact with the skin of the measurement subject;
an amplifier configured to amplify a biopotential detected by the first electrode;
a transmitter configured to modulate a carrier wave according to the biopotential amplified by the amplifier and wirelessly transmit the modulated signal to the other sensor device;
a receiver configured to demodulate the modulated signal transmitted from the other sensor device and extract information on the biopotential;
a reference potential generator configured to generate a reference potential for the amplifier based on the biopotential amplified by the amplifier and the biopotential output from the receiver; and
a correction unit configured to correct at least one of an oscillation frequency of a voltage-controlled oscillator used for modulation by the transmitter and an oscillation frequency of a voltage-controlled oscillator used for demodulation by the receiver.
(Supplementary Note 2) The biosignal measurement system according to supplementary note 1, wherein
each of the first sensor device and the second sensor device further includes:
a transmitting antenna for wirelessly transmitting the modulated signal output from the transmitter to the other sensor device; and
a receiving antenna for receiving the modulated signal transmitted from the other sensor device.
Supplementary Note 3) The biosignal measurement system according to supplementary note 1, wherein
each of the first sensor device and the second sensor device further includes:
a second electrode for transmitting the modulated signal output from the transmitter to the other sensor device via a body of the measurement subject; and
a third electrode for receiving the modulated signal from the other sensor device via the body of the measurement subject.
(Supplementary Note 4) The biosignal measurement system according to supplementary note 1, wherein
the correction unit includes a variable capacitor capable of changing at least one of the oscillation frequency of the voltage-controlled oscillator used for modulation by the transmitter and the oscillation frequency of the voltage-controlled oscillator used for demodulation by the receiver.
(Supplementary Note 5) The biosignal measurement system according to supplementary note 1, wherein
each of the first sensor device and the second sensor device further includes:
a first cable connector for connecting the transmitter of its own sensor device and the receiver of the other sensor device by wiring; and
a second cable connector for connecting the receiver of its own sensor device and the transmitter of the other sensor device by wiring.
(Supplementary Note 6) The biosignal measurement system according to any one of supplementary notes 1 to 5, wherein
the correction unit of each of the first sensor device and the second sensor device is provided to correct the oscillation frequency of the voltage-controlled oscillator used for demodulation by the receiver of its own sensor device,
wherein the oscillation frequency of the voltage-controlled oscillator used for demodulation by the receiver of its own sensor device is adjusted by the correction unit of its own sensor device so that a first potential applied to the transmitter of the other sensor device for modulating the carrier wave and a second potential output from the receiver of its own sensor device in response to the modulated signal transmitted from the other sensor device coincide with each other.
(Supplementary Note 7) The biosignal measurement system according to any one of supplementary notes 1 to 5, wherein
the correction unit of each of the first sensor device and the second sensor device is provided to correct the oscillation frequency of the voltage-controlled oscillator used for modulation by the transmitter of its own sensor device,
wherein the oscillation frequency of the voltage-controlled oscillator used for modulation by the transmitter of its own sensor device is adjusted by the correction unit of its own sensor device so that a first potential applied to the transmitter of its own sensor device for modulating the carrier wave and a second potential output from the receiver of the other sensor device in response to the modulated signal transmitted from its own sensor device coincide with each other.
(Supplementary Note 8) The biosignal measurement system according to any one of supplementary notes 1 to 5, wherein
the correction unit of each of the first sensor device and the second sensor device includes
a first correction unit configured to correct the oscillation frequency of the voltage-controlled oscillator used for demodulation by the receiver of its own sensor device, and
a second correction unit configured to correct the oscillation frequency of the voltage-controlled oscillator used for modulation by the transmitter of its own sensor device, wherein
the oscillation frequency of the voltage-controlled oscillator used for demodulation by the receiver of its own sensor device is adjusted by the first correction unit of its own sensor device so that a first potential applied to the transmitter of the other sensor device for modulating the carrier wave and a second potential output from the receiver of its own sensor device in response to the modulated signal transmitted from the other sensor device coincide with each other, and, further, in case that the first potential and the second potential do not coincide with each other, when a third potential is applied to the transmitter of its own sensor device to modulate the carrier wave, the oscillation frequency of the voltage-controlled oscillator used for modulation by the transmitter of the other sensor device is adjusted by the second correction unit of the other sensor device according to a result of a comparison between a fourth potential output from the receiver of the other sensor device in response to the modulated signal transmitted from its own sensor device and a predetermined threshold voltage.

### INDUSTRIAL APPLICABILITY

Embodiments of the present invention can be applied to techniques for measuring biosignals.

### REFERENCE SIGNS LIST

1a, 1b Sensor device,
2 Biosignal generation device,
101a, 101b, 109a′, 109b′, 110a′, 110b′ Electrode,
102a, 102b Amplifier,
103a, 103b AD converter,
104a, 104b Wireless transmitter,
105a, 105b FM transmitter,
106a, 106b FM receiver,
107a, 107b Reference potential generator,
108a, 108b Power source,
109a, 109b Transmitting antenna,
110a, 110b Receiving antenna,
111a, 111b, 112a, 112b Cable connector,
200 Wireless receiver,
201 Calculation unit,
202 Storage,
1051a Crystal oscillator,
1060a VCO,
1052a, 1061a Phase comparator,
1053a, 1062a Low-pass Filter,
1063a Amplifier,
1054a and 1054b, 1064a, 1064b Correction unit.

1-8. (canceled)

9. A biosignal measurement system comprising:
a first sensor device configured to be attached to one of a right-side portion or a left-side portion of a measurement subject; and
a second sensor device configured to be attached to the other of the right-side portion or the left-side portion, wherein
each of the first sensor device and the second sensor device includes:
a first electrode configured to be in contact with skin of the measurement subject;
an amplifier configured to amplify a biopotential detected by the first electrode;
a transmitter configured to modulate a carrier wave according to the biopotential amplified by the amplifier to generate a modulated signal, and wirelessly transmit the modulated signal to the other sensor device;
a receiver configured to demodulate the modulated signal transmitted from the other sensor device and extract information on the biopotential;
a reference potential generator configured to generate a reference potential for the amplifier based on the biopotential amplified by the amplifier and the biopotential output from the receiver; and
a correction circuit configured to correct at least one of an oscillation frequency of a voltage-controlled oscillator used for modulation by the transmitter and an oscillation frequency of a voltage-controlled oscillator used for demodulation by the receiver.

10. The biosignal measurement system according to claim 9, wherein
each of the first sensor device and the second sensor device further includes:
a transmitting antenna for wirelessly transmitting the modulated signal output from the transmitter to the other sensor device; and
a receiving antenna for receiving the modulated signal transmitted from the other sensor device.

11. The biosignal measurement system according to claim 9, wherein
each of the first sensor device and the second sensor device further includes:
a second electrode for transmitting the modulated signal output from the transmitter to the other sensor device via a body of the measurement subject; and
a third electrode for receiving the modulated signal from the other sensor device via the body of the measurement subject.

12. The biosignal measurement system according to claim 9, wherein
the correction circuit includes a variable capacitor capable of changing at least one of the oscillation frequency of the voltage-controlled oscillator used for modulation by the transmitter and the oscillation frequency of the voltage-controlled oscillator used for demodulation by the receiver.

13. The biosignal measurement system according to claim 9, wherein
each of the first sensor device and the second sensor device further includes:
a first cable connector for connecting the transmitter of its own sensor device and the receiver of the other sensor device by wiring; and
a second cable connector for connecting the receiver of its own sensor device and the transmitter of the other sensor device by wiring.

14. The biosignal measurement system according to claim 9, wherein the correction circuit of each of the first sensor device and the second sensor device is provided to correct the oscillation frequency of the voltage-controlled oscillator used for demodulation by the receiver of its own sensor device,
wherein the correction circuit of its own sensor device adjusts the oscillation frequency of the voltage-controlled oscillator used for demodulation by the receiver of its own sensor device so that a first potential applied to the transmitter of the other sensor device for modulating the carrier wave and a second potential output from the receiver of its own sensor device in response to the modulated signal transmitted from the other sensor device coincide with each other.

15. The biosignal measurement system according to claim 9, wherein
the correction circuit of each of the first sensor device and the second sensor device is provided to correct the oscillation frequency of the voltage-controlled oscillator used for modulation by the transmitter of its own sensor device,
wherein the correction circuit of its own sensor device adjusts the oscillation frequency of the voltage-controlled oscillator used for modulation by the transmitter of its own sensor device so that a first potential applied to the transmitter of its own sensor device for modulating the carrier wave and a second potential output from the receiver of the other sensor device in response to the modulated signal transmitted from its own sensor device coincide with each other.

16. The biosignal measurement system according to claim 9, wherein
the correction circuit of each of the first sensor device and the second sensor device includes
a first correction circuit configured to correct the oscillation frequency of the voltage-controlled oscillator used for demodulation by the receiver of its own sensor device, and
a second correction circuit configured to correct the oscillation frequency of the voltage-controlled oscillator used for modulation by the transmitter of its own sensor device,
wherein the first correction circuit of its own sensor device adjusts the oscillation frequency of the voltage-controlled oscillator used for demodulation by the receiver of its own sensor device so that a first potential applied to the transmitter of the other sensor device for modulating the carrier wave and a second potential output from the receiver of its own sensor device in response to the modulated signal transmitted from the other sensor device coincide with each other, and, in case that the first potential and the second potential do not coincide with each other,
the second correction circuit of the other sensor device adjusts, when a third potential is applied to the transmitter of its own sensor device to modulate the carrier wave, the oscillation frequency of the voltage-controlled oscillator used for modulation by the transmitter of the other sensor device according to a result of a comparison between a fourth potential output from the receiver of the other sensor device in response to the modulated signal transmitted from its own sensor device and a predetermined threshold voltage.

17. A biosignal measurement system comprising:
a first sensor device configured to be attached to one of a right-side portion or a left-side portion of a measurement subject; and
a second sensor device configured to be attached to the other of the right-side portion or the left-side portion, wherein
each of the first sensor device and the second sensor device includes:
a first electrode configured to be in contact with skin of the measurement subject;
an amplifier configured to amplify a biopotential detected by the first electrode;
a transmitter configured to modulate a carrier wave according to the biopotential amplified by the amplifier to generate a modulated signal, and wirelessly transmit the modulated signal to the other sensor device;
a receiver configured to demodulate the modulated signal transmitted from the other sensor device and extract information on the biopotential; and
a reference potential generator configured to generate a reference potential for the amplifier based on the biopotential amplified by the amplifier and the biopotential output from the receiver.

18. A biosignal measurement system comprising:
a first sensor device configured to be attached to one of a right-side portion or a left-side portion of a measurement subject; and
a second sensor device configured to be attached to the other of the right-side portion or the left-side portion, wherein
each of the first sensor device and the second sensor device includes:
a first electrode configured to be in contact with skin of the measurement subject;
an amplifier configured to amplify a biopotential detected by the first electrode;
a transmitter configured to modulate a carrier wave according to the biopotential amplified by the amplifier to generate a modulated signal, and wirelessly transmit the modulated signal to the other sensor device;
a receiver configured to demodulate the modulated signal transmitted from the other sensor device and extract information on the biopotential; and
a correction circuit configured to correct at least one of an oscillation frequency of a voltage-controlled oscillator used for modulation by the transmitter and an oscillation frequency of a voltage-controlled oscillator used for demodulation by the receiver.

19. The biosignal measurement system according to claim 17, wherein
each of the first sensor device and the second sensor device further includes:
a transmitting antenna for wirelessly transmitting the modulated signal output from the transmitter to the other sensor device; and
a receiving antenna for receiving the modulated signal transmitted from the other sensor device.

20. The biosignal measurement system according to claim 17, wherein
each of the first sensor device and the second sensor device further includes:
a second electrode for transmitting the modulated signal output from the transmitter to the other sensor device via a body of the measurement subject; and
a third electrode for receiving the modulated signal from the other sensor device via the body of the measurement subject.

21. The biosignal measurement system according to claim 17, wherein
each of the first sensor device and the second sensor device further includes:
a first cable connector for connecting the transmitter of its own sensor device and the receiver of the other sensor device by wiring; and
a second cable connector for connecting the receiver of its own sensor device and the transmitter of the other sensor device by wiring.

22. The biosignal measurement system according to claim 18, wherein
the correction circuit includes a variable capacitor capable of changing at least one of the oscillation frequency of the voltage-controlled oscillator used for modulation by the transmitter and the oscillation frequency of the voltage-controlled oscillator used for demodulation by the receiver.

23. The biosignal measurement system according to claim 18, wherein the correction circuit of each of the first sensor device and the second sensor device is provided to correct the oscillation frequency of the voltage-controlled oscillator used for demodulation by the receiver of its own sensor device,
wherein the correction circuit of its own sensor device adjusts the oscillation frequency of the voltage-controlled oscillator used for demodulation by the receiver of its own sensor device so that a first potential applied to the transmitter of the other sensor device for modulating the carrier wave and a second potential output from the receiver of its own sensor device in response to the modulated signal transmitted from the other sensor device coincide with each other.

24. The biosignal measurement system according to claim 18, wherein
the correction circuit of each of the first sensor device and the second sensor device is provided to correct the oscillation frequency of the voltage-controlled oscillator used for modulation by the transmitter of its own sensor device,
wherein the correction circuit of its own sensor device adjusts the oscillation frequency of the voltage-controlled oscillator used for modulation by the transmitter of its own sensor device so that a first potential applied to the transmitter of its own sensor device for modulating the carrier wave and a second potential output from the receiver of the other sensor device in response to the modulated signal transmitted from its own sensor device coincide with each other.

25. The biosignal measurement system according to claim 18, wherein
the correction circuit of each of the first sensor device and the second sensor device includes
a first correction circuit configured to correct the oscillation frequency of the voltage-controlled oscillator used for demodulation by the receiver of its own sensor device, and
a second correction circuit configured to correct the oscillation frequency of the voltage-controlled oscillator used for modulation by the transmitter of its own sensor device,
wherein the first correction circuit of its own sensor device adjusts the oscillation frequency of the voltage-controlled oscillator used for demodulation by the receiver of its own sensor device so that a first potential applied to the transmitter of the other sensor device for modulating the carrier wave and a second potential output from the receiver of its own sensor device in response to the modulated signal transmitted from the other sensor device coincide with each other, and, in case that the first potential and the second potential do not coincide with each other,
the second correction circuit of the other sensor device adjusts, when a third potential is applied to the transmitter of its own sensor device to modulate the carrier wave, the oscillation frequency of the voltage-controlled oscillator used for modulation by the transmitter of the other sensor device according to a result of a comparison between a fourth potential output from the receiver of the other sensor device in response to the modulated signal transmitted from its own sensor device and a predetermined threshold voltage.

26. The biosignal measurement system according to claim 9, wherein each of the first sensor device and the second sensor device further includes a motion sensor configured to detect movement of the measurement subject, and wherein the transmitter is further configured to modulate the carrier wave according to motion data detected by the motion sensor.

27. The biosignal measurement system according to claim 9, wherein each of the first sensor device and the second sensor device further includes a temperature sensor configured to measure a skin temperature of the measurement subject, and wherein the reference potential generator is further configured to adjust the reference potential based on the measured skin temperature.

28. The biosignal measurement system according to claim 9, wherein each of the first sensor device and the second sensor device is configured to dynamically adjust a transmission power of the transmitter based on a signal strength of the modulated signal received from the other sensor device.
